(19) United States
(12) Patent Application Publication
(10) Pub. No.: US 2019/0150412 A1
(43) Pub. Date: May 23, 2019

(54) METHODS FOR INCREASING THE RATE OF GENETIC PROGRESS AND GENERATING INBRED LINES IN NON-HUMAN MAMMALS USING GAMETES DERIVED FROM EMBRYOS

(71) Applicant: Inguran, LLC, Navasota, TX (US)

(72) Inventor: Claas Heuer, Heidmoor (DE)

(73) Assignee: Inguran, LLC, Navasota, TX (US)

(21) Appl. No.: 16/195,552

(22) Filed: Nov. 19, 2018

Related U.S. Application Data
(60) Provisional application No. 62/588,817, filed on Nov. 20, 2017.

(51) Int. Cl. A01K 67/027; C12Q 1/6876

(52) U.S. Cl. CPC A01K 67/027; A01K 2227/101; C12Q 2600/124; C12Q 1/6876

(57) ABSTRACT

The invention encompasses methods of increasing the rate of genetic progress or generating inbred lines in a non-human mammalian species comprising the use of gametes derived from embryos of the non-human mammalian species.

### REFERENCE TO RELATED APPLICATIONS

This application claims the benefit of U.S. Patent Application No. 62/588,817 filed Nov. 20, 2018. The entire disclosure of which is incorporated herein by reference.

### BACKGROUND OF THE INVENTION

While assisted reproductive technologies, such as in vitro fertilization, as well as improved methods of phenotypic and genotypic evaluation, have allowed breeders to increase the rate of genetic progress in animals, such gains have been limited to date. Additionally, the ability to produce inbred lines for use in crossbreeding in animal species is time consuming and difficult. Accordingly, there exists a need in animal breeding to increase the rate of genetic progress, as well as to expedite and improve the creation of inbred lines.

### SUMMARY OF THE INVENTION

One embodiment of the invention encompasses a method of fertilizing an egg of a non-human mammalian species, comprising genotyping a plurality of embryos of the non-human mammalian species; selecting a first embryo from the plurality of embryos based on an estimated breeding value (EBV) or genotypic value of the first embryo; deriving eggs from the first embryo; fertilizing the eggs with sperm cells to produce a second plurality of embryos; genotyping the second plurality of embryos; and selecting a second embryo from the second plurality of embryos based on an EBV or genotypic value of the second embryo. A further embodiment comprises the additional step of deriving a sperm cell or an egg from the second embryo. An even further embodiment comprises the additional step of extracting DNA from one or more amniocytes or fibroblasts from the first embryo in vivo, wherein the first embryo remains viable.

An additional embodiment of the invention encompasses a method of fertilizing an egg of a non-human mammalian species, comprising genotyping a plurality of embryos of the non-human mammalian species; selecting a first embryo from the plurality of embryos based on an EBV or genotypic value of the first embryo; deriving sperm cells from the first embryo; fertilizing eggs with the sperm cells to produce a second plurality of embryos; genotyping the second plurality of embryos; and selecting a second embryo from the second plurality of embryos based on an EBV or genotypic value of the second embryo. A further embodiment comprises the additional step of deriving a sperm cell or an egg from the second embryo. An even further embodiment comprises the additional step of extracting DNA from one or more amniocytes or fibroblasts from the first embryo in vivo, wherein the first embryo remains viable.

Another embodiment of the invention encompasses a method of generating a line in a non-human mammalian species, comprising deriving a sperm cell and an egg from a first embryo of the non-human mammalian species; and fertilizing the egg with the sperm cell to produce a second embryo. A further embodiment comprises the additional step of selecting the first embryo based on an EBV, genotypic value or gamete variance, of the first embryo. A yet further embodiment comprises the additional steps of deriving a sperm cell and an egg from the second embryo; fertilizing the egg derived from the second embryo with the sperm cell derived from the second embryo to produce a third embryo. An even further embodiment comprises the additional step of extracting DNA from one or more amniocytes or fibroblasts from the first embryo in vivo, wherein the first embryo remains viable.

The invention, in another embodiment, also encompasses a method of generating a line in a non-human mammalian species, comprising providing a first embryo of the non-human mammalian species and a second embryo of the non-human mammalian species, wherein the first embryo and the second embryo are full-sibs, half-sibs or first cousins, or alternatively, share a common ancestor within the last five generations; deriving a gamete from the first embryo; deriving a gamete from the second embryo; and using the gamete derived from the first embryo and the gamete derived from the second embryo in in vitro fertilization to produce a third embryo. A further embodiment comprises the step of selecting the first embryo and the second embryo from a plurality of embryos based on an EBV, genotypic value, or gamete variance, of the first embryo and an EBV, genotypic value, or gamete variance, of the second embryo. An even further embodiment comprises the additional step of extracting DNA from one or more amniocytes or fibroblasts from the first embryo and the second embryo in vivo, wherein the first embryo and the second embryo remain viable.

In any of the above embodiments, a step of deriving a gamete from an embryo may comprise i) obtaining or deriving an embryonic stem cell from the embryo or ii) deriving an induced pluripotent stem cell from a fibroblast from the embryo.

The various embodiments of the invention may be applied to, or comprise, individuals or species of non-human mammals, and the invention should be understood not to be limited to the species of non-human mammals described by the specific examples within this application. Rather the specific examples within this application are intended to be illustrative of the varied and numerous species of non-human mammals to which the methods of the invention may be applied. Embodiments of the invention, for example, encompass animals having commercial value for meat or dairy production such as swine, ovine, bovine, equine, deer, elk, buffalo, or the like (naturally the mammals used for meat or dairy production may vary from culture to culture). They also encompass various domesticated non-human mammalian species such as canines and felines, as well as primates, including but not limited to chimpanzees, and gorillas, as well as whales, dolphins and other marine mammals. In particular embodiments of any of the above disclosed embodiments, the non-human mammalian species comprises bovids.

### BRIEF DESCRIPTION OF THE DRAWINGS

FIG. 1 shows a schematic of breeding program in which gametes derived from embryos are combined with amniotic or fetal tissue sampling.

FIG. 2 shows that if one uses gametes derived from embryos over multiple generations, genetic progress (dG) decreases over each subsequent generation.

FIG. 3 shows the relationship between continuous mating of related individuals (half sibs, full sibs, and an individual with itself (i.e., selfing)) and the development of inbreeding over generations.

FIG. 4 shows an embodiment of the invention comprising a crossbreeding program using gametes derived from embryos.

FIG. 5 shows the time needed to generate inbred lines using current biotechnology and breeding schemes compared to a breeding program that uses gametes derived from embryos.

### DETAILED DESCRIPTION OF THE INVENTION

The invention encompasses the use of gametes generated or derived from embryonic stem cells or primordial germ cells to increase genetic progress in a genetic nucleus, line, breed or herd, or to create inbred lines within a species.

Within a genetic nucleus, (or line or herd), once selected, parents that produce the next generation are mated with one another, while avoiding matings between closely related individuals, with the goal of increasing the genetic merit of the next generation. An increase in the genetic merit of the next generation constitutes genetic progress. An increase in genetic merit, in this context, means that for a given trait or set of traits, the individuals in the successive generation will express the desired trait or set of traits more strongly than their parents. With respect to undesirable traits, an increase in genetic merit means the individuals in the successive generation will express the trait or set of traits less strongly than their parents.

Genetic change, including desirable genetic change (i.e., genetic progress per year), (“dG”) can be measured as the difference between the average genetic level of all progeny born in one year and all progeny born the following year. The difference is the result of selected parents having higher genetic merit than the average genetic merit of all the selection candidates (the animals available for selection as parents of the next generation). In ideal conditions, this depends upon the heritability (h²) of the trait and the difference between the average performance of selected parents and that of selection candidates. The heritability of a trait (h²) is the proportion of observable differences (phenotypic variance, σ²P) in a trait between individuals within a population that is due to additive genetic (A), as opposed to environmental (E), differences (h²=σ²_{A}/σ²_{P}=σ²_{A}/(σ²_{A}+σ²_{E})). The difference between the average performance of selected parents and that of all selection candidates (of which the selected parents are a subset) is also known as the selection differential.

The genetic progress per year is the result of genetic superiority of selected males and of selected females. This is expressed in the following equation:

dG={(R_{IH}*i)ₘₐₗₑₛ+(R_{IH}*i)_{females}}*σ_{H}/(Lₘₐₗₑₛ+L_{females}),

Where, R=the accuracy of selection, i=the selection intensity, σ_{H}=genetic variation and L=generation interval, for male or female parents.
H=breeding goal that combines genetic merit (g) of the traits (1 to m) that need to be produced weighted by the economic values (v) of the traits (H=v₁g₁+v₂g₂+ . . . +vₘgₘ). The economic value is positive if selection is for larger phenotypic values and negative if selection is for smaller phenotypic values.
I=an index that combines all the trait information on the individual and its relatives and is the best estimate of the value of H for the individual.

As used herein, “breeding value” generally constitutes a value twice the average deviation of an animal's offspring group for a given trait from the population mean. A “parent average” generally constitutes the average of an animal's parents' breeding values, or the sum of the parents' predicted transmitting abilities (PTAs). “Genetic markers” generally constitute identifiable (i.e., typable) regions on the genome for which there is variation in the population. In Holstein dairy cattle breeding, for example, it is of importance to rank animals according to a breeding value. A genetic evaluation can be expressed as either a PTA or an estimated breeding value (EBV). Both are measures of performance relative to a base population, with an individual's PTA simply being one half of the EBV. A PTA indicates the difference in performance that can be expected from an animal's daughters relative to that base; an EBV is the genetic merit of the animal itself relative to the base and, therefore, is twice the PTA. Genomic EBVs (GEBVs) and genomic PTAs (GPTAs) are estimated breeding values and predicted transmitting abilities that incorporate genomic data, including genomic relationships, as commonly derived from single nucleotide polymorphism (SNP) data. As used herein, the terms “EBV” and “PTA” encompass GEBV and GPTA, respectively. The term “breeding value” as used herein encompasses EBVs. The term “genotypic value” encompasses an individual's breeding value plus non-additive genetic effects, such as dominance and epistasis.

In a large population, the selection intensity depends upon how many animals are tested and how many are selected—the lower the proportion selected the higher the selection intensity and the larger the genetic progress, all else being equal. Thus, in order to maximize genetic progress, one should rank all tested animals based on the GEBV, for example, and then select the minimum number of top males and females required to maintain the line, breed and/or herd size and to avoid inbreeding problems. This ensures that the average GEBV of selected animals is substantially higher than the average GEBV of all animals tested. In particular through the use of artificial insemination (AI), one needs to select fewer males than females and the selection intensity for males is higher than for females.

The generation interval for males (or females) is the average age of male parents (or female parents) when progeny are born. The annual rate of genetic progress depends on the generation interval and on the superiority of the parent's GEBVs compared to that of the selection candidates. In general, males contribute more to the genetic progress per year than the females.

“Line” as used herein refers to animals having a common origin and similar identifying characteristics. “Genetic nucleus” as used herein refers to one or more populations of male and female animals used to generate selection candidates in a breeding program. “Breeding program” as used herein refers to a system for making genetic progress or for the creation of lines in a population of animals. The terms “fetus” and “embryo” are used interchangeably herein.

Gametes Derived from Embryos

One aspect of the invention comprises deriving gametes, both oocytes and sperm, directly from in vitro, or in vivo, embryos. In addition to markedly reducing the maturation age of selection candidates, the invention allows a breeder to greatly reduce or entirely eliminate the need for maintaining and caring for young and adult animals as either selection candidates (i.e., prospective parents of the next generation) or as recipients for embryo transfer. In a particular embodiment of the invention, oocytes or sperm can be derived from either embryonic stem cells (ESCs) or from induced pluripotent stem cells (iPSCs) obtained from an embryo or blastocyst (see, e.g., Hayashi et al., Cell 146, 519-532, 2011; ESC derivation from blastocysts is described in Hayashi et al., Cell 146, 519-532, 2011 and iPSCs derivation from embryonic fibroblasts is described in Hikabe et al., Nature 539, 299-303, 2016; derivation of bovine iPSCs from bovine fetal fibroblasts is described in Talluri et al., Cellular Reprogramming, 17, 131-140, 2015). Once ESCs or iPSCs are derived, they can in turn be used to derive primordial germ cell-like cells (PGCLCs) (see, e.g., Hayashi et al., Cell 146, 519-532, 2011). Finally, PGCLCs can be used to derive sperm (see, e.g., Hayashi et al., Cell 146, 519-532, 2011) or oocytes (see e.g., Hikabe et al., Nature 539, 299-303, 2016), in vitro. In an alternative embodiment, oocytes can be derived from primordial germ cells (PGCs) obtained from an embryo or fetus in accordance with any of the methods known in the prior art (see, e.g., Morohaku et al., PNAS, 113, 9121-9036, 2016).

Use of Gametes Derived from Embryos in Assisted Reproductive Technologies

In certain embodiments of the invention, once gametes are derived from one or more embryos, those gametes are used in IVF, or other assisted reproductive technologies such as intracytoplasmic sperm injection, to generate a subsequent generation of embryos. In a further embodiment, this process is repeated multiple times, with each set of derived gametes used to generate a subsequent generation of embryos.

### Selection of Embryos in a Breeding Program

One aspect of the invention encompasses selecting one or more embryos—rather than reproductively mature animals—as parents of the next generation in a breeding program and thereafter deriving gametes from the selected embryos for use in IVF or other assisted reproductive technologies. One embodiment involves selecting embryos based on one or more breeding values, including EBVs and GEBVs, or genotypic values, of the embryo. For example, in a particular embodiment of the invention, a plurality of embryos is generated, and then each embryo is genomically evaluated and ranked relative to the other embryos on the basis of genetic merit. Embryos with the highest genetic merit are selected and gametes are subsequently derived from those selected embryos for use in assisted reproductive technologies. In certain embodiments, this process can be repeated multiple times serially, with each set of derived gametes used to generate a subsequent generation of embryos. In addition to selecting embryos on the basis of EBVs or genotypic values, one embodiment of the invention also includes selecting an embryo based on its gamete variance. Methods for genomically evaluating and ranking embryos on the basis of genetic merit or gamete variance are described in greater detail below.

Reduction of Generation Interval Using Gametes Derived from Embryos

The production of gametes derived from embryonic stem cells or primordial germ cells allows for the reduction of the age of maturity in non-human mammals, thereby reducing the generation interval and increasing the rate of genetic progress. For example, if an IVF bovine embryo is directly used for the in vitro production of oocytes, the reproduction age for that individual would be around −250 days of age. Alternatively, if the embryo is implanted in a recipient and genomically tested using amniotic fluid cells (e.g., fibroblasts or mesenchymal stem cells) at around day 70 of pregnancy, the reproduction age for that individual would be around −170 days of age. Methods for genomically testing embryos via amniocentesis or tissue sampling are provided in U.S. patent application Ser. No. 15/487,244, which is incorporated by reference herein in its entirety.)

This decrease in reproduction age has an immediate impact on the generation interval, which is the age of the reproducing individuals in a population when the individuals are born that are going to replace them. There are four different channels of selection: Sires of bulls, sires of dams, dams of bulls and dams of dams. Each channel has different selection intensities and generation intervals, which is mainly due to differences in reproduction age and capacity.

While females begin reproducing earlier (as soon as oocytes can be harvested, e.g. some weeks of age), males have a higher reproductive capacity because they generate more gametes. When looking at generation interval, there is higher potential for a decrease on the male side. Typically, in bovines, bulls start to have their semen collected at around 11 months of age, which is when they are able to sire the first embryos.

The minimum generation interval (provided in days) that can be achieved using current technology and gametes derived from embryos can be seen in Table 1.

TABLE 1

| | Dams of Next | Sires of Next |
|---|---|---|
| Technology | Generation | Generation |
| Conventional | 325 | 585 |
| Gametes derived from embryos | 40 | 40 |
| Gametes derived from embryos + amniotic | 110 | 110 |
| or fetal tissue sampling | | |

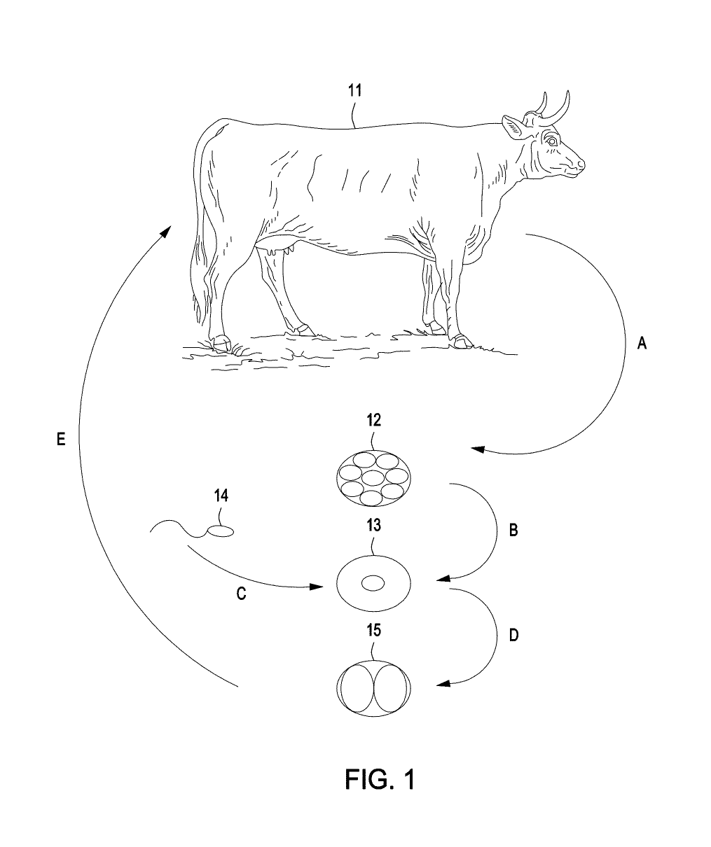

FIG. 1 shows how a cycle in which gametes derived from embryos can be combined with amniotic or fetal tissue sampling. An embryo 12 is obtained from an embryo transfer recipient 11 at step A. Step A comprises either flushing the pregnant embryo transfer recipient 11 to obtain the embryo 12, or alternatively, creating the embryo 12 by i) obtaining an amniotic fluid or fetal tissue sample from the embryo transfer recipient 11 that is pregnant, ii) culturing the amniotic or fetal cells and iii) creating a clone (i.e., embryo 12) via somatic cell nuclear transfer. In step B, one or more oocyte 13 is derived from the embryo 12 in vitro using techniques known in the art. In step C, the oocyte 13 is fertilized with sperm 14 via IVF (or other assisted reproductive technology) to produce a zygote 15, in step D. In step E, the zygote 15 is transferred into the embryo transfer recipient 11. In an alternative embodiment not show, instead of transferring the zygote 15 into the embryo transfer recipient 11 as in step E, the zygote 15 is used to derive an additional oocyte in vitro that is also fertilized with sperm via IVF—this step can be serially repeated as many times as desired by the breeder, with the final embryo being transferred into the embryo transfer recipient 11. In an even further embodiment, one or more embryos of any given generation are selected as parents based on one or more of their breeding values, genotypic values or gamete variance.

The genetic progress (provided in additive genetic standard deviations) that can be achieved using current technology versus gametes derived from embryos can be seen in Table 2.

TABLE 2
Change in genetic progress per year

| | dG for Dams of Next | dG for Sires of Next |
|---|---|---|
| Technology | Generation | Generation |
| Conventional | 1.78 | 0.99 |
| Gametes | 14.46 | 14.46 |
| derived from | | |
| embryos | | |
| Gametes | 5.26 | 5.26 |
| derived from | | |
| embryos + | | |
| amniotic or | | |
| fetal tissue | | |
| sampling (see | | |
| FIG. 1) | | |

Efficiency of Selection when Using Gametes Derived from Embryos Over Multiple Generations

The fast turnover of generations possible when using gametes derived from embryos makes, it necessary to consider the impact on additive genetic variance, accuracy of selection and inbreeding of the selected embryos/animals over time. After multiple generations, the selected embryos/animals will have a fairly large distance to the actively phenotyped population. Impact of using Gametes Derived from Embryos on Additive Genetic Variance

When doing truncation selection using some selection criteria (e.g., “Net Merit $,” a selection index published by the US Department of Agriculture), the additive genetic variance in the population of selection candidates is:

σ2gsel=(1−kr2)σ2g,

where σ2g is the additive genetic variance in the population before selection and k=i(i−t), with i being the selection intensity and t the truncation point of the normal distribution (Bulmer, 1971; Deckers, 2014—Course notes). r is the accuracy of selection. The breeding values of the offspring is the parent average of the parents plus a Mendelian sampling term and the additive genetic variance of these offspring is:

σ2goffspring=0.25σ2gSire+0.25σ2gDam+var(ε).

Using the above equation one can model the change in additive genetic variance over multiple selection cycles. Usually an equilibrium variance is reached at some early stage, but only if inbreeding is being kept at low levels.

Impact of Using Gametes Derived from Embryos on Accuracy of Selection

The next figure that changes by doing selection of animals over several generations is the accuracy of selection. First, the average relationship of the selected animals to the phenotyped population decreases over time, and second, the linkage disequilibrium (LD) structure between quantitative trait loci (QTL) and markers changes, which reduces the accuracy of estimates of marker effects (Habier et al., 2007).

Traditionally one could also argue that the reduction in additive genetic variance results in a reduced heritability which reduces accuracy of selection if the selection criteria is the phenotype. If the selection criteria is an EBV, then accuracy decreases if the resources to estimate the EBVs are kept constant at lower heritabilities.

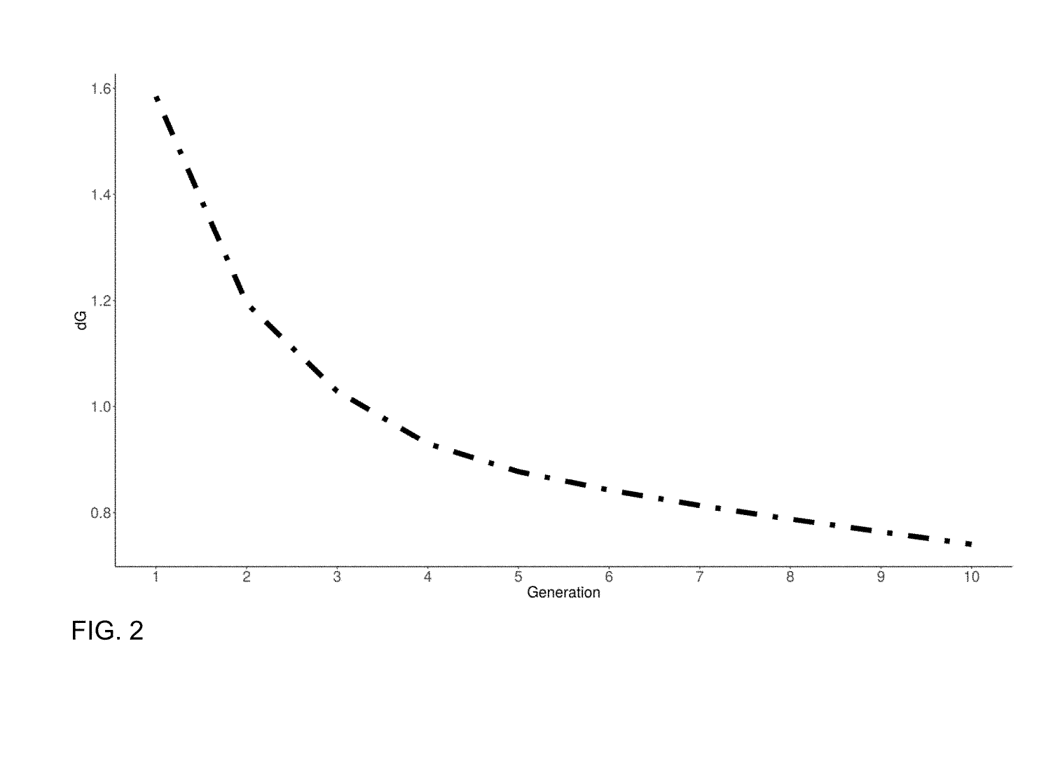

As shown in FIG. 2, if one uses gametes derived from embryos over multiple generations, genetic progress (dG) decreases over each subsequent generation. This is due, at least in part, to a decrease in the accuracy of selection, a decrease in genetic variability, and inbreeding. The projected decrease in genetic progress over generations is based on simulation results in Habier et al., 2007.

### Generating Inbred Lines In Vitro for a Crossbreeding Program

One aspect of the invention is the ability to quickly and efficiently generate inbred lines using gametes (sperm and/or eggs) derived from embryos that are closely related to each other (e.g., full sibs, half sibs or first cousins, or share a common ancestor within the last five generations) or gametes derived from the same embryo (selfing). In each line, the embryo(s) generated from the derived gametes are then used to derive gametes that are used to generate the next generation embryo(s). This step can be repeated as many times as needed in order to generate a desired level of inbreeding. The final step is then to crossbreed those two newly generated inbred lines to generate hybrid embryos as the commercial end product, which are then transferred into recipient animals in order to produce production animals.

The desirability of inbred lines lies in the exploitation of heterosis, which is caused by gene interaction at QTL loci. Heterosis, or hybrid vigor, can arise when mating two inbred and unrelated lines to create hybrid offspring generations. Besides heterosis, generating hybrids as final products to consumers allows protection of genetic progress being made in the inbred lines, while delivering a highly uniform and specialized group of individuals. However, prior to the instant invention, in most mammals, the long gestation length and late age of maturity made it time consuming an inefficient to generate highly inbred animals by mating related individuals.

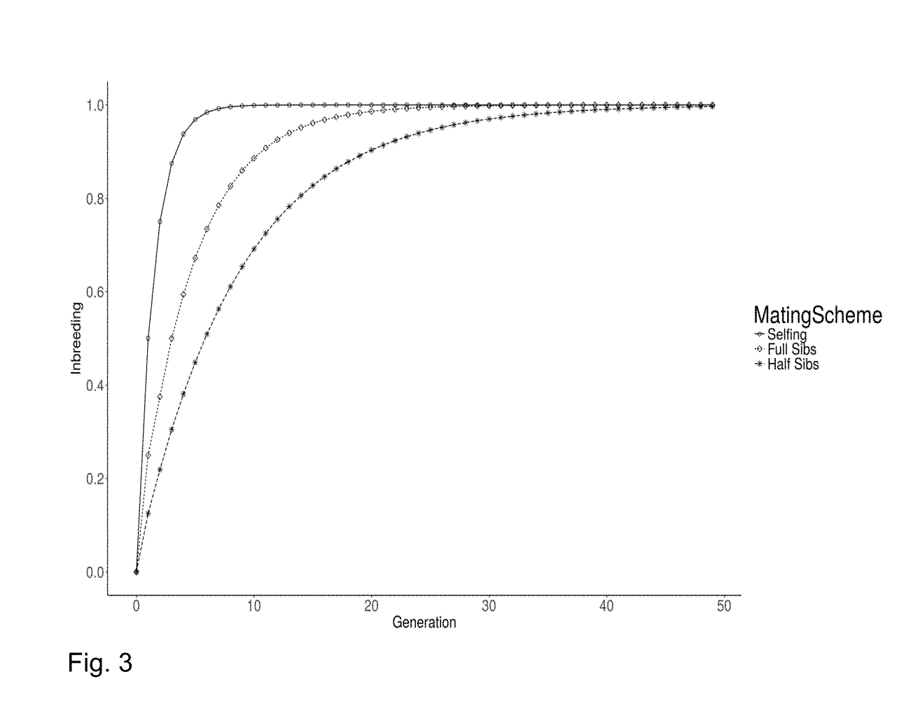

FIG. 3 shows the relationship between continuous mating of related individuals (half sibs, full sibs, and the individual with itself (i.e., selfing)) and the development of inbreeding over generations. Inbreeding fixates alleles and reduces Mendelian sampling variance (as a linear function of the inbreeding coefficient). Hence, the higher the inbreeding of the sire and dam, the more uniform an offspring group is going to be. In the extreme case with inbreeding coefficients of one, all offspring will be identical to one another (i.e., effectively clones). Given the traditional mating schemes, the creation of inbred lines that exceed inbreeding coefficients of, for example, 0.5, was an undertaking that typically took many years prior to the instant invention.

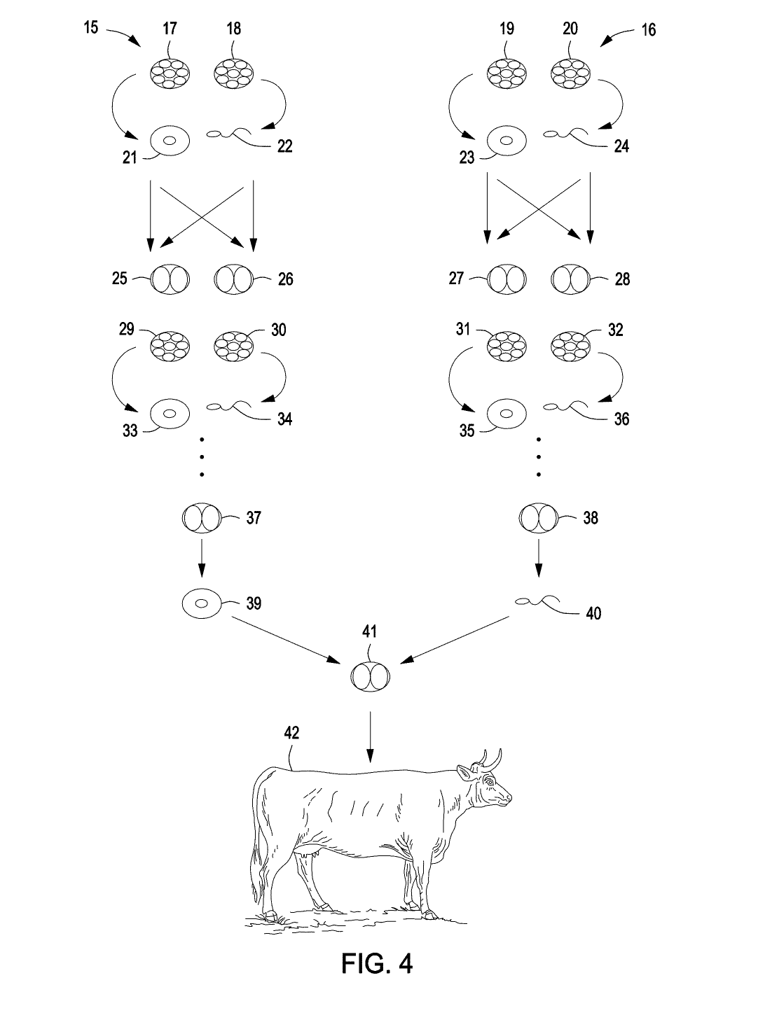

In FIG. 4, the concept of a crossbreeding program using gametes derived from embryos is illustrated. This embodiment of the invention hinges on the creation of inbred lines either completely in vitro, or in conjunction with amniotic or fetal tissue sampling. As an example a two line cross is illustrated. Two separate inbred lines 15 and 16 are established by starting off with two subpopulations (embryos 17 and 18 constitute the first subpopulation and embryos 19 and 20 constitute the second subpopulation) that are extreme for traits of interest and that one would like to combine in the final end product (i.e., hybrid 41).

Referring to FIG. 4, in line 15, embryos 17 and 18 are full sibs, and in line 16, embryos 19 and 20 are full sibs. Gametes 21, 22, 23 and 24 are derived from embryos 17, 18, 19 and 20, respectfully. In line 15, gametes 21 and 22 are combined in an IVF step to produce zygote 25, which develops into embryo 29, and an additional pair of gametes 21 and 22 are combined to produce zygote 26, which develops into embryo 30—embryos 29 and 30 being full sibs. Similarly, in line 16, gametes 23 and 24 are combined in an IVF step to produce zygote 27, which develops into embryo 31, and an additional pair of gametes 23 and 24 are combined to produce zygote 28, which develops into embryo 32—embryos 31 and 32 being full sibs. Gametes 33, 34, 35 and 36 are then derived from embryos 29, 30, 31 and 32, respectively. In one embodiment, this process—i.e., creating full sib embryos, deriving gametes from the embryos and then combining the gametes in an IVF step to produce zygotes—is repeated serially over multiple generations (as many times as desired by the breeder depending on the level of inbreeding desired). Gametes 33 and 34 and gametes 35 and 36 are in turn combined in an IVF step as above to produce zygote 37 in line 15 and zygote 38 in line 16, respectively. Gametes 39 and 40 are then derived from the zygotes 37 and 38, respectively, once they have reached the embryonic stage. In the final step, gametes 39 and 40 are combined in an IVF step to produce the hybrid 41 of line 15 and line 16. The hybrid 41 can then either be transferred to a recipient 42 and/or cloned if desired.

Referring to FIG. 4, in one embodiment not shown, a cell sample from one or more embryos of each generation is collected and genotyped. In this embodiment, only the most genetically superior individuals are selected as parents for the next embryo generation. Alternatively, instead of using genetic merit as the selection criteria, in one embodiment, one could use gamete variance as the selection criteria. Methods for calculating gamete variance are disclosed in U.S. Patent Application No. 65/527,838, which is hereby incorporated by reference in its entirety. In one embodiment, it is desirable to select embryos demonstrating the lowest values for gamete variance, which would be useful for generating inbreeding on a per trait basis.

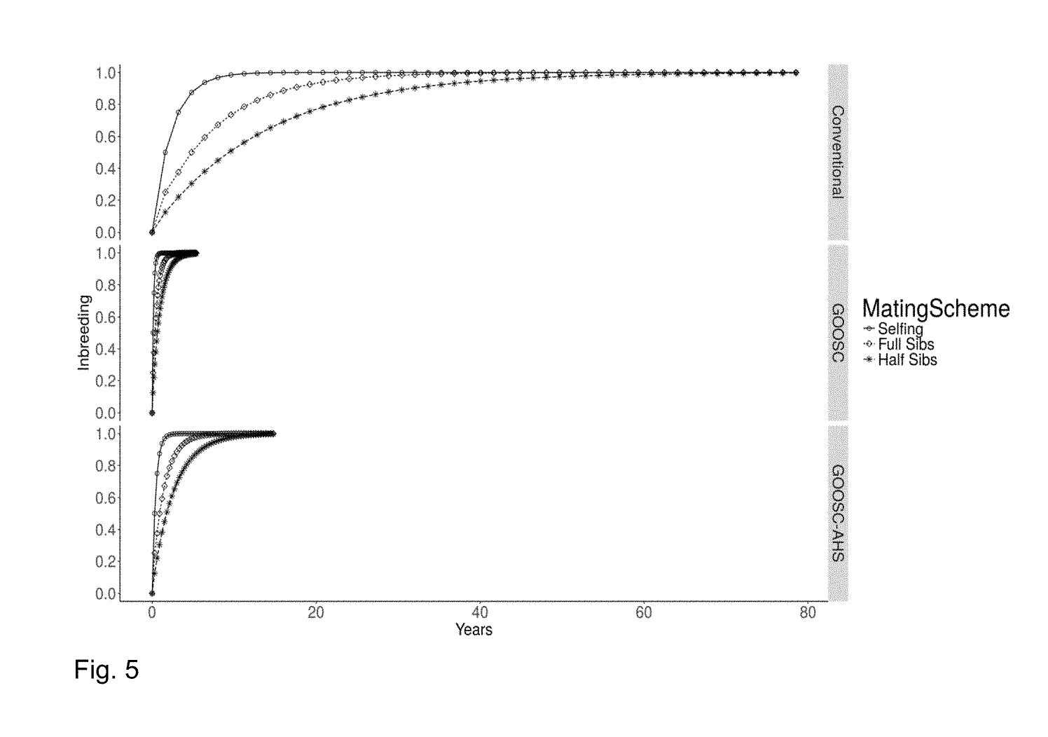

FIG. 5 illustrates the time needed to generate inbred lines using current biotechnology and breeding schemes compared to the use of gametes derived from embryos (“GOOSC”) and gametes derived from embryos in conjunction with amniotic or fetal tissue sampling (“GOOSC-AHS”). (The results from FIG. 3 have been multiplied by the generation intervals given in Table 1 to give the change in inbreeding in years, rather than generations.) As shown, using full sib mating in the conventional case, it takes around 10 years in the best case to achieve an inbreeding coefficient of 0.7, while in 6 months, almost completely inbred individuals can be generated when using gametes derived from embryos.

In contrast to the prior art, the generation of inbred lines using gametes derived from embryos allows a breeder to decrease the response time to market changes. For example, if for economic or political reasons, certain traits become important in the market, it would take many years (even with genomic selection) to achieve recognizable genetic progress in new traits using conventional technology. When using gametes derived from embryos, however, this response time can be dramatically decreased. Using this embodiment of the invention, in a period of less than a year, a breeder can produce animals for the market that are superior by multiple standard deviations compared to the competition.

### Embryo Production In Vivo and In Vitro

In certain embodiments of the invention, embryos may be produced in vivo by traditional methods for synchronized supernumerary follicle production, artificial insemination (AI) and scheduled non-surgical transvaginal catheterized intrauterine embryo recovery. In other aspects of the invention, in vitro produced embryos may be produced in the laboratory by non-typical harvest of oocytes, IVF and embryo culture methodologies. In peripubertal heifers, prophase I immature cumulus oocyte complexes (COCs) are recovered from live standing females by using ultrasound guided transvaginal oocyte recovery (TVOR) system, also referred to as ovum pickup (OPU). In prepubertal heifers, ultrasound guided laparoscopic OPU is employed for COC recovery. When immature COCs are brought into the laboratory, they are placed into typical in vitro maturation (IVM) culture system where the most developmentally capable oocytes undergo spontaneous and programmed meiosis. After an overnight culture period, those oocytes that progress through meiosis I (and accordingly shed their second polar body progressing to metaphase of the second meiotic division) and are morphologically normal (including an intact plasma membrane) are used in IVF. Mature oocytes from individual females are placed into traditional IVF drops and mated to specific sires, using highly screened and accurate sperm capacitation treatments and sperm concentration per oocyte fertilized. Zygotes (day 1) are placed into traditional co-culture system and cultured to uterine stages of development by day 7-8 of culture. Embryos are typically transported to a recipient heifer farm where they are non-surgically transferred. Prior to transfer, embryos may be biopsied or sampled for genetic screening and/or genomic evaluation. Within certain specific stages of embryo development, embryos can be dismantled and used in embryo multiplication procedures and/or cryopreserved for later use. Embryos destined for transfer to synchronized surrogate females are transported to the farm in culture and non-surgically transferred by traditional methods. In certain embodiments, the invention contemplates that recipient females are regularly checked by veterinarians and ongoing pregnancies are monitored on a regular and scheduled basis via transrectal real time ultrasonography.

### Embryo Transfer

Certain embodiments of the invention encompass embryo transfer. The following surgical and non-surgical methods of embryo transfer are provided by way of non-limiting example only.

In cattle, an embryo can be transferred via mid-line abdominal incision, or a flank incision, to a recipient under general anesthesia. Recipients are placed in squeeze chutes that give access to either flank. The corpus luteum is located by rectal palpation and the flank ipsilateral to the corpus luteum is clipped, washed with soap and water, and sterilized with iodine and alcohol. About 60 ml of 2 percent procaine is given along the line of the planned incision. A skin incision is made about 15 cm long, high on the flank, just anterior to the hip. Muscle layers are separated, and the peritoneum is cut. The surgeon inserts a hand and forearm into the incision, locates the ovary, generally about 25 cm posterior to the incision, and visualizes or palpates the corpus luteum. The uterine horn is exteriorized by grasping and stretching with the thumb and forefinger the broad ligament of the uterus, which is located medial to the uterine horn. A puncture wound is made with a blunted needle through the wall of the cranial one-third of the exposed uterine horn. Using about 0.1 ml of medium in a small glass pipette (<1.5 mm outside diameter), the embryo is drawn up from the storage container. The pipette is then inserted into the lumen of the uterus, and the embryo is expelled. The incision is then closed, using two layers of sutures.

Alternatively, a non-surgical method may be used to transfer an embryo in cattle. First, it is necessary to palpate ovaries in order to select the side of ovulation, since pregnancy rates are lowered if embryos are transferred to the uterine horn contralateral to the corpus luteum. Recipients should be rejected if no corpus luteum is present or pathology of the reproductive tract is noted. The next step is to pass the embryo transfer device, e.g., a standard Cassou inseminating gun, through the cervix. The third step of non-surgical transfer is to insert the tip of the instrument into the desired uterine horn ipsilateral to the corpus luteum. The final step of the procedure is to transfer the embryo from a container, such as a straw, into the desired uterine horn using the transfer device.

### Collection of Amniotic Fluid

Certain embodiments of the invention encompass methods of collecting amniotic fluid. Once amniotic fluid is collected, a further aspect includes isolating fetal cells from the amniotic fluid and performing genomic analysis on DNA extracted from the fetal cells. Any method known in the art for collection of amniotic fluid may be used in the invention, including but not limited to trans-vaginal/trans-uterine collection using either ultrasound guided or manual puncture techniques. Additionally, amniotic fluid may be collected at any time during gestation in a mother or embryo transfer recipient, including from day 45 through parturition, or between day 1 to day 10, day 20 to day 30, 30 to day 280, day 40 to day 100, day 50 to day 80, day 60 to day 70, day 70 to day 80, day 80 to day 90, day 90 to day 100, day 100 to day 120, day 70 to day 90, day 75 to day 80, day 75 to day 90, day 70 to day 85, or day 120 to day 280, of gestation.

By way of example, the following collection procedure may be used in the invention. One skilled in the art will know that variations on this method exist and that this method should not be construed to limit the functionality or scope of the current invention. This method is illustrative only.

Obtain a bovine mother, or recipient, with a fetus on day 65 to day 250 of gestation. Administer standard caudal epidural anesthesia with 2% lidocaine. Raise the animals approximately 40 cm at the front using a platform in order to place the reproductive tract back towards the pelvis. Clean and disinfect the vulva region and inside of the vaginal vaults several times with iodine. Trans-rectally retract the uterus with the opposite hand and juxtapose the pregnant horn against the vaginal wall. Insert an ultrasound-transducer covered with a sterile sleeve into the vaginal vault with the aid of light lubrication approximately to the level of the cervix. Aspirate the fetal fluid by intra-vaginal placement of a needle (Ø=1.3 mm, 68 cm length) installed within the body of the ultrasound-transducer and connected to a vacuum-tube blood collection assembly. Ultrasound scanner may be equipped with a 5.0 MHz convex type transducer approximately 1.6 cm wide and 58 cm long. Advance the needle through the vaginal and uterine walls by sharply moving the vacuum tube over a distance of about 3 to 4 cm. If the syringe plunger meets resistance, reposition the needle and take another aspirate. Transfer the aspirate was to a sterile 10 ml test tube, placed on ice, and submit for DNA analysis. Confirm successful needle placement by direct observation of ultrasonography and fetal fluid swirling within the vacuum tube. Fetal viability may be assessed between 7 to 10 days after the aspiration procedure. Imaging of either independent fetal movement or heart beat may be taken as proof of viability.

Another collection method in pregnant cattle encompasses the use of ultrasound-guided transvaginal oocyte recovery (TVOR) equipment, specialized fluid recovery tubing, and adapted filter collection system. In this example, in all cattle destined for amniocentesis, pregnancy is confirmed and fetal sex determined by transrectal ultrasonography at specific periods after embryo transfer, implantation and the completion of organogenesis. By day 45-100, or more specifically day 75-80, of the first trimester of gestation, ultrasound-guided transvaginal oocyte recovery equipment is adapted and used to visualize the entire fetus and amniotic vesicle in a uterine horn during aspiration. Prior to collection, the heifers are restrained in stocks and sedated prior to performing amniocentesis. The veterinary staff performing amniocentesis use complete sterile procedures, including powder free nitrile gloved hands and ethanol sterilization of equipment. To ensure that the area is free of contamination before insertion of the transducer, the rectum is emptied of feces, and under epidural anesthesia the vulva and rectal area of the cow are thoroughly cleaned and scrubbed. The disinfection step is completed by rinsing the vulva and rectal area with Betadine solution and the rinsing and spraying the cleaned area with 70% ethanol. The TVOR equipment is cleaned and sterilized with ethanol immediately prior to its introduction into the vagina and is fitted with a sterile stainless steel single-needle guide. The TVOR equipment is advanced into the vagina, positioned to the left or the right of the cervical os and by means of manipulation per rectum, the pregnant uterine horn is positioned against the probe, avoiding interposition of other tissue in the proposed needle path. The exact location of the amniotic sac is determined by the recognition of fetal body parts, the allantoamniotic and allantochorionic membranes and the uterine wall. When a non-echogenic area representing amniotic fluid is seen on the monitor screen, a sterile needle with a stylette is inserted within the needle guide and advanced penetrating through the vaginal wall, uterus and subsequent fetal membranes. As soon as the tip of the needle is seen to have entered the fetal fluid compartment, the stylette is withdrawn from the needle and the needle is placed inside the amnion of the fetus. An initial 5-10 ml of fetal fluid is aspirated into the tubing and flushed out of the tubing system to reduce or eliminate maternal contamination. An amniocentesis filter is attached to the tubing and an additional 30-40 ml of amniotic fluid is aspirated. During the fluid collection, the pregnant uterine horn is held in the same position, and the exact location of the tip of the needle is guaranteed by its visualization on the ultrasound screen. When samples from more than 1 heifer are collected on the same day, the needle-guide is replaced by a sterile one, and the transducer is thoroughly cleaned and disinfected before being used on the next animal. After collection of amniotic fluid is completed in an animal, the collected fluid in the filter system is placed on ice and transported back to the cell culture laboratory.

Isolating Amniocytes from Amniotic Fluid

The term “amniocytes” as used herein, refers to cells obtained from amniotic fluid, as well as to cells cultured from cells obtained from amniotic fluid. Amniocytes, including fetal fibroblasts and amniotic fluid-derived mesenchymal stem cells (AFMSCs), when used in the present invention may be obtained from, e.g., amniotic fluid from amniocentesis performed for fetal karyotyping, or amniotic fluid obtained at term. For purposes of the invention, amniocytes may be isolated from the amniotic fluid by any method known in the art, e.g., by centrifugation followed by removal of the supernatant.

### Amniocyte Cell Culture

One aspect of the invention encompasses culturing isolated amniocytes. Cultured amniocytes can in turn be used in various applications, including genotyping and for producing clones. By way of example, the following culturing procedure may be used in certain embodiments of the invention. One skilled in the art will know that variations on this method exist and that this method should not be construed to limit the functionality or scope of the current invention. This method is illustrative only.

Amniocytes are centrifuged (200 g, 10 min) at room temperature and the pellet is gently resuspended in Chang medium. Cells are plated into 100 mm gelatinized Petri dishes and left undisturbed. Media is changed every 3-4 days. After 2 weeks in culture, they are trypsinized to disperse cells and allow their growth in a monolayer. Amniocytes are cultured at 37° C. in a humidified 5% CO₂ atmosphere. Cells are passaged at a ratio 1:4 every 5 days until they reach 80% confluence. For subsequent passages, the media is aspirated, washed with PBS, detached with 0.05% trypsine for 5 min at 37° C.

### Isolation and Culture of Amniotic Fluid-Derived Mesenchymal Stem Cells

In certain embodiments of the invention, a two-stage culture method may be used to isolate, culture, and enrich amniotic fluid-derived mesenchymal stem cells (AFMSCs) from amniotic fluid obtained by amniocentesis. Mammalian mesenchymal stem cells are presumptively multipotent cells that have the potential to differentiate into multiple lineages including bone, cartilage, muscle, tendon, ligament fat and a variety of other connective tissues. Morphologically, mesenchymal stem cells in their undifferentiated state are spindle shaped and resemble fibroblasts. Mesenchymal stem cells have been identified mostly in bone marrow, but have also been found in both adult and fetal peripheral blood, fetal liver, fetal spleen, placenta and in term umbilical cord blood. Significantly, mesenchymal stem cells can be found in mammalian amniotic fluid. Under specific culture conditions, mammalian AFMSCs have been induced to differentiate into adipocytes, osteocytes and neuronal cells.

The two-stage culture protocol comprises a first stage of culturing amniocytes, and a second stage of culturing mesenchymal stem cells. The method begins by setting up primary cultures using cytogenetic laboratory amniocytes culture protocol. Non-adhering amniotic fluid cells in the supernatant medium are collected. For culturing mesenchymal stem cells, the non-adhering cells are centrifuged and then plated in a culture flask with an alpha-modified Minimum Essential Medium supplemented with fetal bovine serum. For mesenchymal stem cell growth, the culture is incubated with humidified CO₂.

By way of example, the following specific culturing procedure may be used in certain embodiments of the invention. One skilled in the art will know that variations on this method exist and that this method should not be construed to limit the functionality or scope of the current invention. This method is illustrative only.

For culturing amniocytes, set up four primary in situ cultures in 35 mm tissue culture-grade dishes using Chang medium (Irvine Scientific, Santa Ana, Calif). Collect non-adhering amniotic fluid cells in the supernatant medium on the 5th day after the primary amniocytes culture and keep them until a completion of fetal chromosome analysis.

For culturing mesenchymal stem cells, centrifuge the tube containing the non-adhering cells, then plate them in 5-15 ml of alpha-modified Minimum Essential Medium (α-MEM) supplemented with 10-20% fetal bovine serum (FBS) and 1-20 ng/ml b-FGF in a 25cm² culture flask and incubate at 37° C. with 5% humidified CO₂ for mesenchymal stem cell growth.

Flow cytometry, RT-PCR, and immunocytochemistry may be used to analyze the phenotypic characteristics of the cultured mesenchymal stem cells. Von Kossa, Oil Red O and TuJ-1 stainings may be used to assess the differentiation potentials of the mesenchymal stem cells.

The following additional culture method is presented by way of example only. The invention contemplates sterile technique, including being gloved with non-powder nitrile gloves to process amniotic fluid. In certain embodiments of the invention, the entire process is performed in a cell culture laminar flow biosafety cabinet and only food grade ethanol is used in washing gloved hands whenever needed or possible.

Fluid and amniocytes are aspirated by pipette into 15 ml conical tubes. The collection filter is rinsed with culture medium to remove any adhered cells and repeated as necessary to remove a maximal amount of amniocytes from the filter. The conical tubes are centrifuged until a cell pellet is formed, supernatant is aspirated, and cells are resuspended in cell culture medium. The cell suspension is thoroughly mixed and pipetted into culture wells and/or dishes. Cell cultures are placed into a cell culture incubator and cultured at 38.7C in 5% CO₂/air for 5 days undisturbed. On day 5 of culture, cell culture dishes are removed from culture and cell culture medium and any floating cells are aspirated and placed into 15 ml centrifuge tube. The remaining cells plated on the original cell culture dishes, primarily fetal fibroblasts and AFMSCs are fed with fresh culture medium and placed back into cell culture incubators and cultured until 80-90% confluent. After reaching confluency, the cells are lifted for passage and/or cryopreservation. The aspirated floating amniocytes can be started in amniocyte-specific cell culture or used in fetal diagnostic testing and/or genomic testing and profiling. Both original plated fetal fibroblast cultures and original floating amniocyte cell cultures can be cultured for indefinite passaging and cryopreservation. Cryopreserved fetal fibroblasts and/or amniocytes can be warmed and passaged or used in cloning procedures.

### Fetal and Embryonic Tissue Sampling

In addition, or alternative, to obtaining fetal cells from amniotic fluid, one aspect of the invention also encompasses obtaining a fetal or embryonic tissue or cell sample directly from the fetus or embryo. In one embodiment, the invention encompasses taking an in vivo or in vitro fetal or embryonic tissue or cell sample on day 1, 2, 3, 4, 5, 6, 7, 8, 9, 10, 12, 13, 14, 15, 16, 17, 18, 19, 20, 21, 22, 23, 24, 25, 26, 27, 28, 29 or 30 of gestation or after fertilization, or between day 1 to day 10, day 15 to day 25, day 20 to day 30, day 21 to day 26, day 21 to day 23, day 24 to day 26, day 30 to day 40, day 40 to day 50, day 60 to day 70, day 30 to day 280, day 40 to day 100, day 50 to day 80, day 60 to day 70, day 70 to day 80, day 80 to day 90, day 90 to day 100, day 100 to day 120, day 70 to day 90, day 75 to day 80, day 75 to day 90, day 70 to day 85, or day 120 to day 280, of gestation or after fertilization. In a certain embodiment of the invention, the removed fetal or embryonic cells comprise fibroblasts, stem cells or primordial germ cells. In a more particular embodiment, the removed cells comprise one or more blastomeres, one or more cells from an inner cell mass of a blastocyst or one or more cells from an epiblast layer of a blastocyst. In one embodiment, the fetal or embryonic tissue or cell sample is removed from the fetus or embryo without sacrificing the fetus or embryo, i.e., without affecting the viability of the fetus or embryo. In certain embodiments of the invention, after extraction of fetal or embryonic cells derived from an embryo or fetus, the viable embryo or fetus is developed and produced as offspring subsequent to transfer into a recipient.

Any know method for in vitro fetal or embryonic tissue or cell sampling is contemplated for use with the invention. Additionally any know method for in vivo fetal or embryonic tissue or cell sampling, whether transvaginal, transabdominal, transcervical, or otherwise, is contemplated for use with the invention. The following in vivo fetal or embryonic tissue sampling procedure is presented by way of example only. Briefly, the recipient or mother undergoes a preliminary ultrasonographic examination to confirm gestational age, determine fetal viability, diagnose multiple pregnancy, diagnose fetal structural abnormalities, determine fetal lie, and/or locate the placenta. The recipient or mother is sedated and the abdomen prepared with an iodine-based solution and alcohol. The skin is infiltrated with a 1% lidocaine hydrochloride solution for local anesthesia. A 16.5 gauge thin-walled needle is introduced into the amniotic cavity under continuous ultrasonographic guidance. The biopsy needle is then inserted into the fetus. Once the needle is in the fetus, a syringe is attached to aspirate cells into the biopsy needle. The tissue is removed from the needle by flushing with saline solution. An ultrasound examination may be done immediately after the procedure to assess fetal viability. Fetal fibroblasts are subsequently isolated from the tissue sample by a standard trypsinization procedure using Try-LE (Life Technologies).

Alternatively, another embodiment of the invention contemplates collecting fetuses or embryos from recipients or mothers on day 1, 2, 3, 4, 5, 6, 7, 8, 9, 10, 12, 13, 14, 15, 16, 17, 18, 19, 20, 21, 22, 23, 24, 25, 26, 27, 28, 29 or 30 of gestation, or between day 1 to day 10, day 10 to day 15, day 15 to day 25, day 20 to day 30, day 21 to day 26, day 21 to day 23, day 24 to day 26, day 30 to day 40, day 40 to day 50, day 60 to day 70, day 30 to day 280, day 40 to day 100, day 50 to day 80, day 60 to day 70, day 70 to day 80, day 80 to day 90, day 90 to day 100, day 100 to day 120, day 70 to day 90, day 75 to day 80, day 75 to day 90, day 70 to day 85, or day 120 to day 280, of gestation for genomic analysis by any known technique in the art, including flushing. By way of example only, the following flushing procedure can be utilized in the invention. On day 20 to 26 of gestation, recipient cows are confined in a cattle chute and given an epidural block of 4-6 ml lidocaine. A sterile 20 gauge Foley catheter is inserted through the cervix into the entrance of a horn near the uterine body and the cuff of the catheter is inflated to keep the catheter in position. Vigro Complete Flushing Solution (Bioniche Animal Health) is flushed through the uterus non-surgically while gently squeezing out from the horn of the uterus towards the cervix to expel the fetuses and/or embryos and membranes with the flushing medium. This flushing procedure is then repeated on the contralateral uterine horn. The flushed medium is collected via gravity flow in an EQ way filter (SPI, Canton, Tex.). The flushed contents and filter are taken to the laboratory and carefully washed onto a square grid search dish under a laminar flow hood, and the fetuses and/or embryos are collected using a stereomicroscope. Thereafter, the fetuses and/or embryos are disaggregated and individual fibroblast cell lines can be established from the fetuses and/or embryos. Cells can be passaged once and cultured from 3 to 5 days to obtain homogenous fibroblast populations. Alternatively, stem cells or primordial germ cells can be isolated and cultured from the collected fetuses and/or embryos in accordance with methods in the prior art.

### Genotyping DNA

In one aspect of the invention, extracted and/or amplified DNA from embryonic or fetal cells (e.g., stem cells, primordial germ cells, amniocytes, fibroblasts and mesenchymal stem cells) may be genotyped using SNP arrays or chips, which are readily available for various species of animals from companies such as Illumina and Affymetrix. For purposes of the invention, the term “genotyping” includes, but is not limited to, obtaining SNP and/or copy number variation (CNV) data from DNA. For purposes of the invention, the term “genotype” includes, but is not limited to, SNP and/or copy number variation (CNV) data obtained from DNA. Low density and high density chips are contemplated for use with the invention, including SNP arrays comprising from 3,000 to 800,000 SNPs. By way of example, a “50K” SNP chip measures approximately 50,000 SNPs and is commonly used in the livestock industry to establish genetic merit or genomic estimated breeding values (GEBVs). In certain embodiments of the invention, any of the following SNP chips may be used: BovineSNP50 v1 BeadChip (Illumina), Bovine SNP v2 BeadChip (Illumina), Bovine 3K BeadChip (Illumina), Bovide LD BeadChip (Illumina), Bovine HD BeadChip (Illumina), Geneseek® Genomic Profiler™ LD BeadChip, or Geneseek® Genomic Profiler™ HD BeadChip.

Determining GEBVs from SNP Data

The basis, and algorithm, for using SNPs in determining GEBVs is found in Meuwissen et al., “Prediction of total genetic value using genome-wide dense marker maps,” Genetics 157, 1819 1829 (2001), which is incorporated by reference herein in its entirety. Implementation of genomic data in predictions for desirable traits is found in Van Raden, “Efficient Methods to Compute Genomic Predictions,” Dairy Science 91, 4414 4423 (2008), which is incorporated by reference herein in its entirety.

Livestock in the United States are often ranked using selection indexes that incorporate data related to various commercially important traits. With the advent of genomic testing, genomic data is now commonly used to predict these traits. To calculate an animal's score for a genomic selection index, one must first calculate the animal's GEBVs for each trait in the index, which can be accomplished using the teachings in Meuwissen et al. and VanRaden, above. Next, one determines the economic weight for each trait in the index. Finally, to determine the animal's score for the selection index, multiply each trait's GEBV by its economic weight and then sum all of these values together.

A genomic index commonly used in the United States for dairy cattle is the Genomic Total Performance Index (GTPI®), which is comprised of the following traits: protein; feed efficiency; dairy form; feet and legs composite; somatic cell score; daughter calving ease; fat; udder composite; productive life; fertility index; and daughter stillbirth. In certain embodiments, feed efficiency is equal to the dollar value of milk produced less feed costs for extra milk and less extra maintenance costs, and the fertility index is a function of heifer conception rate, cow conception rate and daughter pregnancy rate. In other embodiments of the invention, GEBV is used to determine Genomic Predicted Transmitting Ability (GPTA).

By way of example, in addition to determining a GEBV for a trait, the presence or absence of any of the following diseases and/or traits can be detected using SNP data or genomic data: Demetz syndrome; white heifer disease; Weaver syndrome (haplotype BHW); haplotype HHD; haplotype HH1; lethal brachygnathia trisomy syndrome; haplotype HH0; bovine hereditary cardiomyopathy; bovine dilated cardiomyopathy; neuronal ceroid lipofuscinosis; bovine chondrodysplastic dwarfism; notched ears/nicked ears; idiopathic epilepsy; bilateral convergent strabismus with exophthalmos; haplotype BHP; haplotype HHP; haplotype JHP; neuropathic hydrocephalus/water head; congenital hypotrichosis and anodontia defect/ectodermal dysplasia; ichthyosis fetalis; lethal trait A46/bovine hereditary zinc deficiency; Marfan Syndrome; double muscling; multiple ocular defects; bovine ocular squamous cell carcinoma; pink tooth; posterior paralysis/hind-limb paralysis; haplotype BHM; bovine spongiform encephalopathy/mad cow disease; mule foot disease (haplotype HEIM); myophosphorylase deficiency; dropsy; black/red coat color (haplotype HBR; haplotype HHR); BAND3 deficiency; Charolais ataxia; bovine spinal dysmyelination (haplotype BHD); Dun coat color in Dexter cattle; bovine familial convulsions and ataxia; bulldog calf; simmental hereditary thrombopathy; GHRD; renal tubular dysplasia (RTD)/chronic interstitial nephritis; Hereford white face; haplotype HHC; developmental duplications; black kidney; cardiomyopathy/Japanese black cattle; crooked tail syndrome; congenital pseudomyotonia; bovine hereditary arthrogyposis multiplex congentia; belted; Syndrome d'Hypoplasie Généralisée Capréoliforme; fawn calf syndrome; bovine neonatal pancytopenia; rat-tail syndrome; cheilognathoschisis; German White Fleckvieh syndrome; haplotype JH1; paunch calf syndrome; acorn calf disease/congenital joint laxity and dwarfism; haplotype HH2; haplotype HH3; haplotype HH4; Holstein bull-dog dwarfism; haplotype AH1; haplotype HH5; haplotype JH2; and lethal arthrogyposis syndrome.

### Cloning

An additional aspect of the invention encompasses cloning embryos and/or fetuses that have been genomically evaluated using the techniques disclosed herein. Cloning is generally understood to be the creation of a living animal/organism that is essentially genetically identical to the unit or individual from which it was produced. In those embodiments of the invention that encompass cloned embryos and/or fetuses, any method by which an animal can be cloned that is known in the art can be utilized. Thus, it is contemplated that cloned embryos and cloned fetuses are produced by any conventional method, for instance including the cloning techniques described herein, as well as those described in international patent application PCT/US01/41561. In one aspect of the invention, a basis for cloning an embryo or a fetus is its genomic merit. In a further aspect, the embryo or fetus's genetic merit is determined by genomic analysis as disclosed herein.

Cloning of embryos by nuclear transfer has been developed in several species. This technique generally involves the transfer of a cell nucleus (obtained from a donor cell) into an enucleated cell, for instance, a metaphase II oocyte. This oocyte has the ability to incorporate the transferred nucleus and support development of a new embryo (Prather et al., Biol. Reprod 41:414-418, 1989; Campbell et al., Nature 380:64-66, 1996; Wilmut et al., Nature 385:810-813, 1997). Morphological indications of this re-programming are the dispersion of nucleoli (Szollosi et al., J. Cell Sci. 91:603-613, 1988) and swelling of the transferred nucleus (Czolowska et al., 1984; Stice and Robl, Biol. Reprod 39:657-664, 1988; Prather et al., J. Exp. Zool. 225:355-358, 1990; Collas and Robl. Biol. Reprod 45:455-465, 1991). The most conclusive evidence that the oocyte cytoplasm has the ability to re-program is the birth of offspring from nuclear transplant embryos in several species, including sheep (Smith and Wilmut, Biol. Reprod. 40:1027 1035, 1989; Campbell et al., Nature 380:64-66, 1996; Wells et al., Biol. Reprod. 57:385-393, 1997), cattle (Wells et al., Biol. Reprod. 60:996-1005, 1999; Kato et al., Science 282:2095-2098, 1998; Prather et al., Biol. Reprod. 37:859-866, 1987; Bondioli et al., Theriogenology 33:165-174, 1990), pigs (Prather et al., Biol. Reprod. 41:414-418, 1989) and rabbits (Stice and Robl, Biol. Reprod. 39:657-664, 1988).

Although the foregoing invention has been described in some detail, one of ordinary skill in the art will understand that certain changes and modifications may be practiced within the scope of the claims.

What we claim is:

1. A method of increasing the rate of genetic progress in a non-human mammalian species, comprising
genotyping a plurality of embryos of the non-human mammalian species;
selecting a first embryo from the plurality of embryos based on an estimated breeding value (EBV) or genotypic value of the first embryo;
deriving eggs from the first embryo;
fertilizing the eggs with sperm cells to produce a second plurality of embryos;
genotyping the second plurality of embryos; and
selecting a second embryo from the second plurality of embryos based on an EBV or genotypic value of the second embryo.

2. The method of claim 1, further comprising the step of deriving a sperm cell or an egg from the second embryo.

3. The method of claim 1, further comprising the step of extracting DNA from one or more amniocytes or fibroblasts from the first embryo in vivo, while maintaining the viability of the first embryo.

4. The method of claim 1, wherein the step of deriving eggs from the first embryo comprises i) obtaining or deriving an embryonic stem cell from the first embryo or ii) deriving an induced pluripotent stem cell from a fibroblast from the first embryo.

5. The method of claim 1, wherein the non-human mammalian species comprises bovids.

6. A method of increasing the rate of genetic progress in a non-human mammalian species, comprising
genotyping a plurality of embryos of the non-human mammalian species;
selecting a first embryo from the plurality of embryos based on an EBV or genotypic value of the first embryo;
deriving sperm cells from the first embryo;
fertilizing eggs with the sperm cells to produce a second plurality of embryos;
genotyping the second plurality of embryos; and
selecting a second embryo from the second plurality of embryos based on an EBV or genotypic value of the second embryo.

7. The method of claim 6, further comprising the step of deriving a sperm cell or an egg from the second embryo.

8. The method of claim 6, further comprising the step of extracting DNA from one or more amniocytes or fibroblasts from the first embryo in vivo, while maintaining the viability of the first embryo.

9. The method of claim 6, wherein the step of deriving sperm cells from the first embryo comprises i) obtaining or deriving an embryonic stem cell from the first embryo or ii) deriving an induced pluripotent stem cell from a fibroblast from the first embryo.

10. The method of claim 6, wherein the non-human mammalian species comprises bovids.

11. A method of generating a line in a non-human mammalian species, comprising deriving a sperm cell and an egg from a first embryo of the non-human mammalian species; and fertilizing the egg with the sperm cell to produce a second embryo.

12. The method of claim 11, further comprising the step of selecting the first embryo based on an EBV, genotypic value, or gamete variance of the first embryo.

13. The method of claim 11, further comprising the step of extracting DNA from one or more amniocytes or fibroblasts from the first embryo in vivo, while maintaining the viability of the first embryo.

14. The method of claim 11, further comprising the steps of
deriving a sperm cell and an egg from the second embryo;
fertilizing the egg derived from the second embryo with the sperm cell derived from the second embryo to produce a third embryo.

15. The method of claim 11, wherein the step of deriving the sperm cell and the egg from the first embryo comprises i) obtaining or deriving an embryonic stem cell from the first embryo or ii) deriving an induced pluripotent stem cell from a fibroblast from the first embryo.

16. The method of claim 11, wherein the non-human mammalian species comprises bovids.

17. A method of generating a line in a non-human mammalian species, comprising providing a first embryo of the non-human mammalian species and a second embryo of the non-human mammalian species, wherein the first embryo and the second embryo are full-sibs, half-sibs or first cousins, or share a common ancestor within the last five generations;
deriving a gamete from the first embryo;
deriving a gamete from the second embryo; and
using the gamete derived from the first embryo and the gamete derived from the second embryo in in vitro fertilization to produce a third embryo.

18. The method of claim 17, further comprising the step of extracting DNA from one or more amniocytes or fibroblasts from the first embryo and the second embryo in vivo, while maintaining the viability of the first embryo and the second embryo.

19. The method of claim 17, further comprising the step of selecting the first embryo and the second embryo from a plurality of embryos based on an EBV, genotypic value, or gamete variance, of the first embryo and an EBV, genotypic value or gamete variance, of the second embryo.

20. The method of claim 17, wherein the step of deriving a gamete from the first embryo comprises i) obtaining or deriving an embryonic stem cell from the first embryo or ii) deriving an induced pluripotent stem cell from a fibroblast from the first embryo, and wherein the step of deriving a gamete from the second embryo comprises i) obtaining or deriving an embryonic stem cell from the second embryo or ii) deriving an induced pluripotent stem cell from a fibroblast from the second embryo.

21. The method of claim 17, wherein the non-human mammalian species comprises bovids.
